(19) United States
(12) Patent Application Publication
(10) Pub. No.: US 2011/0306561 A1
(43) Pub. Date: Dec. 15, 2011

(54) COMPLEMENT C3a DERIVED DIMERIC PEPTIDES AND USES THEREOF

(75) Inventors: Israel Pecht, Rehovot (IL); Anna Erdei, Budapest (HU); Anat Eitan, Rehovot (IL)

(73) Assignee: Yeda Research And Devlopment Co. Ltd., Rehovot (IL)

(21) Appl. No.: 13/148,950

(22) PCT Filed: Feb. 11, 2010
(86) PCT No.: PCT/IL10/00126

§ 371 Date: Aug. 11, 2011

Related U.S. Application Data
(60) Provisional application No. 61/151,859, filed on Feb. 12, 2009.

(51) Int. Cl. A61K 38/10; A61P 11/02; A61P 1/12; A61P 17/00; A61P 1/00; A61P 1/08; A61P 37/08; A61P 11/00

(52) U.S. Cl. 514/18.7; 514/21.4

(57) ABSTRACT

Peptide compositions comprising a dimeric peptide which combines two peptide monomers, each independently comprising the amino acid sequence:

Asp-X1-X2-Asn-Tyr-Ile-Thr-X3

wherein:
X1 is selected from the group consisting of Cys and a Cys derivative;
X2 is selected from the group consisting of Cys and a Cys derivative; and
X3 is selected from the group consisting of Arg and Glu-Leu-Arg, provided that at least one of X1 and X2 is Cys,
   whereby a mol percentage of the dimeric peptide in the composition is at least 50 mol percents, or at least 99 mol percents, are disclosed. Further disclosed are processes of preparing such peptide compositions and uses thereof in the treatment of allergic disorders.

### FIELD AND BACKGROUND OF THE INVENTION

The present invention, in some embodiments thereof, relates to dimeric peptides based on the C-terminal sequence of human complement C3a. The dimeric peptides inhibit secretory responses of mast cells and basophils and accordingly may be useful in the treatment of allergic disorders, such as asthma.

Mast cells and basophils play a central role in inflammatory and immediate hypersensitivity reactions. Clustering of the type 1 Fcε receptors (FcεRI) present in the plasma membranes of mast cells and basophils initiates a coupling network culminating in the secretion of inflammatory mediators including histamine, serotonin, proteases, leukotriens and several cytokines. The molecular mechanism of signal transduction initiated by FcεRI clustering has been intensively studied over the past few years. Lyn, a src family protein tyrosine kinase (PTK) interacts with the β subunit of the receptor complex and undergoes phosphorylation and activation as a result of FcεRI clustering. Recruitment of Lyn to the immunoreceptor tyrosine-based activation motif (ITAM)-phosphorylated receptor subunits inter-alia results in activation of Syk PTK which in turn causes phospholipase C-γ (PLC-γ) activation, hydrolysis of phosphatidyl-inositide-4,5-bisphosphate (PIP2) and a transient rise in free cytosolic [Ca2+]i. This in turn induces activation of protein kinase C culminating eventually in the cells' secretory response.

Mast cell progenitors represent a single lineage, giving rise, upon migration into different tissues to two distinct phenotypes; the so-called serosal (connective tissue type) mastocytes residing in serosal cavities, in the skin and respiratory tract; and the mucosal type mast cells found mainly in regions exposed to potential pathogens such as the gastrointestinal mucosal surfaces. Nevertheless, mast cell tissue-dependent differentiation is reversible; fibroblast derived factors change mucosal type mast cells into serosal ones, while IL-3 favors the mucosal phenotype. Besides tissue distribution, life span and mediator content of their intracellular granules are also different. Both types express FcεRI on their cell membrane, clustering of which provokes the secretory response.

In contrast to the FEεRI-mediated triggering of mastocytes, only serosal mast cells respond to the ‘peptidergic’ stimuli. The latter cells are experimentally modeled by rat peritoneal or human skin mast cells. The peptidergic stimulus is triggered by exposure to polyamines or cationic peptides such as substance P, or the complement activation products C3a and C5a (Mousli et al., Immunopharmcol. 27: 1-11, 1995). These complement-derived anaphylatoxins are among the most potent peptidergic activators of (serosal) mast cells' secretory response. In contrast, mucosal mast cells, such as the rat basophilic leukemia cell line (RBL-2H3) do not respond to such cationic peptides. It was demonstrated that C3a and some of its derivatives inhibit the IgE-mediated degranulation of RBL-2H3 cells, while C5a has no effect on this process (Erdei et al. Int. Immunol. 7: 1433-1439, 1995; Erdei et al. Immunol. Lett. 68: 79-82, 1999).

However, the native intact C3a is not suitable as a potential anti-allergic drug primarily because it contains an activating motif that makes it anaphylatoxic to serosal mast cells, i.e., it is capable of inducing mediator secretion from mast cells.

U.S. Pat. No. 6,682,740 to Erdei et al. discloses peptides corresponding partially or entirely to positions 50-77 of the sequence of human complement-derived peptide C3a and analogs thereof capable of inhibiting IgE-mediated triggering and/or the FcεRI-induced secretory response of mucosal mast cells.

International Application Publication No. WO 2007/013083 to Pecht et al. discloses peptides derived from and corresponding partially to the amino acid sequence at positions 55-64 of human complement component C3a and uses thereof for inhibiting FcεRI-induced secretory response of mucosal-type mast cells, serosal-type mast cells and basophils.

### SUMMARY OF THE INVENTION

There remains an unmet need for compounds having improved efficacy in preventing or treating allergic disorders.

Embodiments of the present invention relate to multimeric peptides, particularly dimeric peptides, comprising peptide monomers derived from and corresponding partially to the amino acid sequence at positions 55-64 of human complement component C3a useful for inhibit secretory responses of mast cells and basophils and accordingly may be useful for the treatment of allergic disorders, such as asthma. As discussed hereinabove, the present inventors have previously designed and successfully prepared and practiced monomeric peptides derived from and corresponding partially to the amino acid sequence at positions 55-64 of human complement component C3a (see, WO 2007/013083). These monomeric peptides were prepared and used while maintaining a reduced form thereof, namely, while not being subjected to oxidizing conditions where formation of disulfide bridges are formed so as to produce dimeric and other multimeric peptides.

The present inventors have tested the effect of combining peptide monomers derived from and corresponding partially to the amino acid sequence at positions 55-64 of human complement component C3a into multimeric peptides, and have uncovered that dimeric peptides formed from such monomeric peptides exhibit improved water solubility and stability as compared to their building monomeric peptides, and hence exhibit improved bioavailability. Moreover, the present inventors have uncovered that dimeric peptides which are prepared such that a single dimeric species is obtained exhibit an improved biological activity as compared to both their building peptide monomers and a mixture of dimeric peptides.

As demonstrated in the Examples section that follows, it has been uncovered that the dimeric peptides disclosed herein are highly effective in inhibiting FcεRI-mediated activation of mast cells and basophils. It has further been uncovered that the dimeric peptides described herein are capable of reducing allergic symptoms such as passive systemic anaphylaxis in animal models.

According to an aspect of embodiments of the present invention there is provided a peptide composition comprising a dimeric peptide, the dimeric peptide comprising two peptide monomers, each independently comprising the amino acid sequence:

Asp-X1-X2-Asn-Tyr-Ile-Thr-X3

wherein:
X1 is selected from the group consisting of Cys and a Cys derivative;
X2 is selected from the group consisting of Cys and a Cys derivative; and
X3 is selected from the group consisting of Arg and Glu-Leu-Arg,
provided that at least one of X1 and X2 is Cys,

wherein a mol percentage of the dimeric peptide in the composition is at least 50 mol percents.

According to some embodiments of the invention, at least one of the peptide monomers comprises the amino acid sequence in which one of X1 and X2 is the Cys derivative.

According to some embodiments of the invention, each of the peptide monomers comprises the amino acid in which one of X1 and X2 is the Cys derivative.

According to some embodiments of the invention, the Cys derivative is devoid of a free thiol group.

According to some embodiments of the invention, the Cys derivative is selected from the group consisting of a protected Cys and Ser.

According to some embodiments of the invention, the Cys derivative is Ser. Optionally, the Cys derivative is any other amino acid that does not affect properties and activity of the peptide. An exemplary amino acid is Thr.

According to some embodiments of the invention, each the peptide monomers independently has an amino acid sequence selected from the group consisting of Asp-Cys-Ser-Asn-Tyr-Ile-Thr-Arg as set forth in SEQ ID NO:4; and Asp-Ser-Cys-Asn-Tyr-Ile-Thr-Arg as set forth in SEQ ID NO:5.

According to some embodiments of the invention, at least one of the peptide monomers comprises the amino acid sequence in which each of X1 and X2 is Cys.

According to some embodiments of the invention, each of the peptide monomers comprises the amino acid in which each of X1 and X2 is Cys.

According to some embodiments of the invention, each of the peptide monomers comprises the amino acid sequence Asp-Cys-Cys-Asn-Tyr-Ile-Thr-Arg as set forth in SEQ ID NO:3.

According to some embodiments of the invention, the two peptide monomers are linked to one another by at least one disulfide bond.

According to some embodiments of the invention, the two peptide monomers are linked to one another by a single disulfide bond.

According to some embodiments of the invention, the disulfide bond is an intermolecular disulfide bond formed between two of the Cys residues.

According to some embodiments of the invention, at least one of the two peptide monomers consists of the amino acid sequence.

According to some embodiments of the invention, each of the two peptide monomers consists of the amino acid sequence.

According to some embodiments of the invention, a mol percentage of the dimeric peptides in the composition is at least 90 mol percents.

According to some embodiments of the invention, a mol percentage of the dimeric peptides in the composition is at least 99 mol percents.

According to some embodiments of the invention, a mol percentage of a multimeric peptide, a monomeric peptide and/or a dimeric peptide other than the dimeric peptide is lower than 1 mol percent.

According to some embodiments of the invention, the dimeric peptide is characterized by an ordered beta sheet structure.

According to some embodiments of the invention, the dimeric peptide comprises and anti-allergic activity.

According to another aspect of embodiments of the present invention there is provided a process of preparing the peptide composition described herein, the process comprising:

reacting two monomeric peptides, each independently comprising the amino acid sequence:

Asp-X1-X2-Asn-Tyr-Ile-Thr-X3

wherein:
X1 is selected from the group consisting of Cys and a Cys derivative;
X2 is selected from the group consisting of Cys and a Cys derivative; and
X3 is selected from the group consisting of Arg and Glu-Leu-Arg, provided that at least one of X1 and X2 is Cys,

in the presence of an oxidizing agent, thereby producing the peptide composition.

According to some embodiments of the invention, the reacting is performed under conditions that favor intermolecular interactions between the monomeric peptides.

According to some embodiments of the invention, the reacting is performed in the presence of a solvent.

According to some embodiments of the invention, at least one of the monomeric peptides comprises an amino acid sequence in which one of X1 and X2 is a Cys derivative.

According to some embodiments of the invention, the Cys derivative is Ser.

According to some embodiments of the invention, the Cys derivative is a protected Cys.

According to some embodiments of the invention, the process is further comprising, prior to reacting the two monomeric peptides with the oxidizing agent:

reacting a monomeric peptide which comprises an amino acid sequence in which X1 and X1 are each Cys with a cysteine protecting group, to thereby obtain the monomeric peptide is which each of X1 and X2 is the protected Cys.

According to some embodiments of the invention, the process is further comprising, prior to reacting the two monomeric peptides with the oxidizing agent:

selectively removing the cysteine protecting group, to thereby obtain a monomeric peptide is which one of X1 and X2 is the protected Cys.

According to some embodiments of the invention, the process is further comprising, prior to reacting the two monomeric peptides with the oxidizing agent:

reacting monomeric peptides which comprises an amino acid sequence in which X1 and X1 are each Cys with a cysteine protecting group, under conditions that favor selective protection, to thereby obtain the peptide monomer is which one of X1 and X2 is the protected Cys.

According to some embodiments of the invention, the oxidizing agent is iodine.

According to an aspect of embodiments of the present invention there is provided a peptide composition as described herein which is being characterized for use in the treatment of an allergic disorder.

According to an aspect of embodiments of the present invention there is provided a pharmaceutical composition comprising the peptide composition as described herein, and a pharmaceutically acceptable carrier.

According to some embodiments of the invention, the pharmaceutical composition is being packaged in a packaging material an identified in print, in or on, the packaging material, for use in the treatment of an allergic disorder.

According to an aspect of embodiments of the present invention there is provided a method of treating an allergic disorder comprising administering to a subject in need thereof a therapeutically effective amount of the peptide composition as described herein, thereby treating the allergic disorder.

According to an aspect of embodiments of the present invention there is provided a use of the peptide composition as described herein in the manufacture of a medicament for treating an allergic disorder.

According to some embodiments of the invention, the allergic disorder results from an IgE- or IgG-mediated (Type I or Type III) hypersensitivity and/or FcεRI- or FcεR-induced secretory response.

According to some embodiments of the invention, the allergic disorder is mediated by a cell type selected from the group consisting of mucosal-type mast cells, serosal-type mast cells and basophils.

According to some embodiments of the invention, the allergic disorder is selected from the group consisting of allergic rhinitis, pulmonary diseases, allergic dermatosis, allergic conjunctivitis, gastrointestinal allergies, cramping, nausea, vomiting, diarrhea, irritable bowel disease, ophthalmic allergies, cheilitis, vulvitis, and anaphylaxis.

According to some embodiments of the invention, the pulmonary disease is asthma.

According to some embodiments of the invention, the allergic dermatosis is urticaria.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D are graphs illustrating the experimentally obtained optical activity of the generated peptide (red line) and the calculated data obtained using a program that evaluates the amount of ordered structure (green line). Y values represent circularly polarized light (degrees) absorption and X values represent wavelength (nm).

FIGS. 2A-F are graphs illustrating the inhibitory effect of the dimeric peptides (FIGS. 2D-F) compared to their peptide monomers (FIGS. 2B-C) and full length peptide (FIG. 2A) on mast cell secretion. Data are expressed as fraction of enzyme (β-hexosaminidase) secreted upon a suboptimal stimulus by antigen (DNP₁₁-BSA) from RBL-2H3 cells. The average values of the inhibitory activity results of 6 independent experiments (each performed in triplicates) are presented.

FIG. 3 is a bar graph illustrating inhibition by peptides of degranulation of human basophils as monitored by flow-cytometric measurement of the cell-surface CD63 expression upon FcεRI-clustering. Data are expressed as percentage of activation-induced CD63 expression obtained after a suboptimal stimulus. Results shown are average values of 6 independent experiments. The final concentration of the employed peptides was 200 μM.

FIG. 4 is a bar graph illustrating the inhibitory effect of peptides of histamine-release induced by an in vivo challenge in mice. Data shown are results of 3 experiments.

FIG. 5 is a bar graph illustrating the inhibitory capacity of monomeric or dimeric peptides with sequences derived from human complement component C3a on FcεRI-mediated secretory response of RBL-2H3 cells assayed by β-hexosaminidase activity.

FIG. 6 illustrates the inhibition of CD63 expression by monomeric or dimeric peptides with sequences derived from human complement component C3a after FcεRI-clustering on human basophils. The numbers above the small columns indicate the number of donors tested.

FIG. 7 depicts the effect of monomeric or dimeric peptides with sequences derived from human complement component C3a on histamine release in mice exposed to passive systemic anaphylaxis.

### DESCRIPTION OF SPECIFIC EMBODIMENTS OF THE INVENTION

The present invention, in some embodiments thereof, relates to dimeric peptides based on the C-terminal sequence of human complement C3a. The dimeric peptides inhibit secretory responses of mast cells and basophils and as such may be used to treat allergic disorders such as asthma.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In the search for peptides capable of inhibiting mast cell activity, the present inventors synthesized and characterized novel peptides based on the complement activation product C3a.

In an attempt to generate peptides with well-defined structures, while considering the presence of two cysteine residues in previously reported peptide monomers derived from and corresponding partially to human complement component C3a, (e.g., C3a9), the present inventors synthesized C3a-based peptide derivatives that comprise a single cysteine residue. Bound by this restriction, only a single dimer species can be produced upon thiol oxidation, thereby eliminating the potential problem of peptides that could be transformed into ill-defined structures by e.g., non-selective dimer formation, intramolecular reactions and/or oligomer formation due to the two cysteine thiol side chains present in earlier peptides (e.g. C3a9).

The dimeric peptides disclosed in the present invention are derived from the known human complement component C3a, a 77-mer polypeptide as set forth in SEQ ID NO:1 of the sequence:

Ser-Val-Gln-Leu-Thr-Glu-Lys-Arg-Met-Asp-Lys-Val-
Gly-Lys-Tyr-Pro-Lys-Glu-Leu-Arg-Lys-Cys-Cys-Glu-
Asp-Gly-Met-Arg-Glu-Asn-Pro-Met-Arg-Phe-Ser-Cys-
Gln-Arg-Arg-Thr-Arg-Phe-Ile-Ser-Leu-Gly-Glu-Ala-
Cys-Lys-Lys-Val-Phe-Leu-Asp-Cys-Cys-Asn-Tyr-Ile-
Thr-Glu-Leu-Arg-Arg-Gln-His-Ala-Arg-Ala-Ser-His-
Leu-Gly-Leu-Ala-Arg.

Particularly, the dimeric peptides disclosed herein are derived from and corresponding partially to the amino acid sequence of positions 55-64 of human complement peptide C3a of the sequence: Asp-Cys-Cys-Asn-Tyr-Ile-Thr-Glu-Leu-Arg (also referred to as C3a7) as set forth in SEQ ID NO:2, to analogs, chemical derivatives, and salts thereof capable of inhibiting mast cell and/or basophil activation. More particularly, the dimeric peptides disclosed herein are derived from and corresponding partially to the amino acid sequence of positions 55-64 of human complement peptide C3a of the sequence: Asp-Cys-Cys-Asn-Tyr-Ile-Thr-Arg (also referred to as C3a9) as set forth in SEQ ID NO:3, to analogs, chemical derivatives, and salts thereof capable of inhibiting mast cell and/or basophil activation.

While reducing the present invention to practice, the present inventors prepared dimeric peptides of C3a9, have further synthesized two peptides with modified C3a sequences, namely DSCNYITR (SEQ ID NO: 5) and DCSNYITR (SEQ ID NO: 4) each having a single cysteine residue, and generated well-defined dimers therefrom.

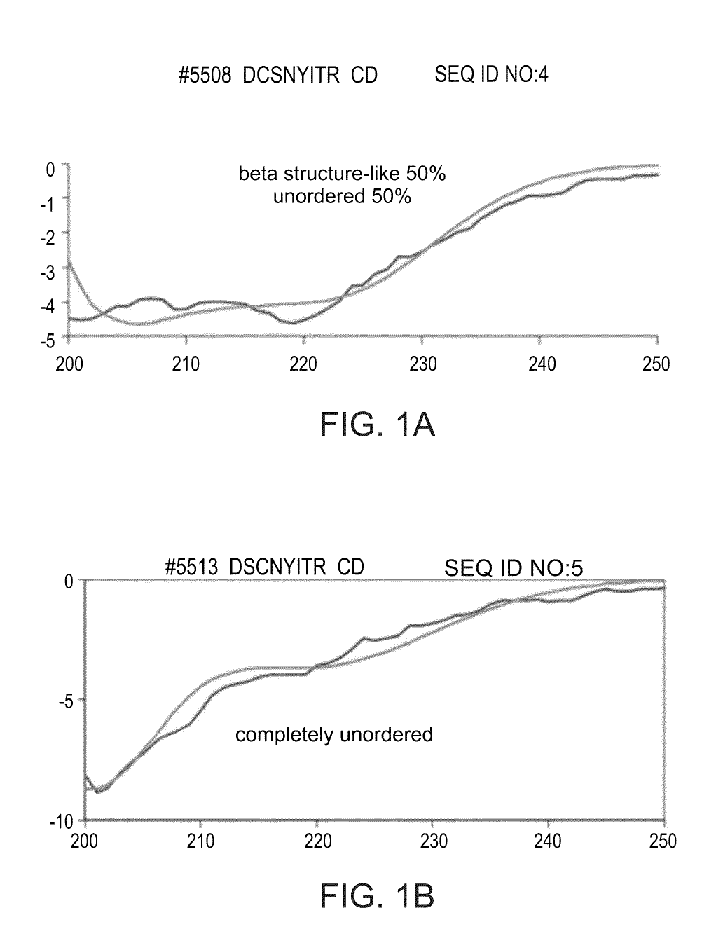

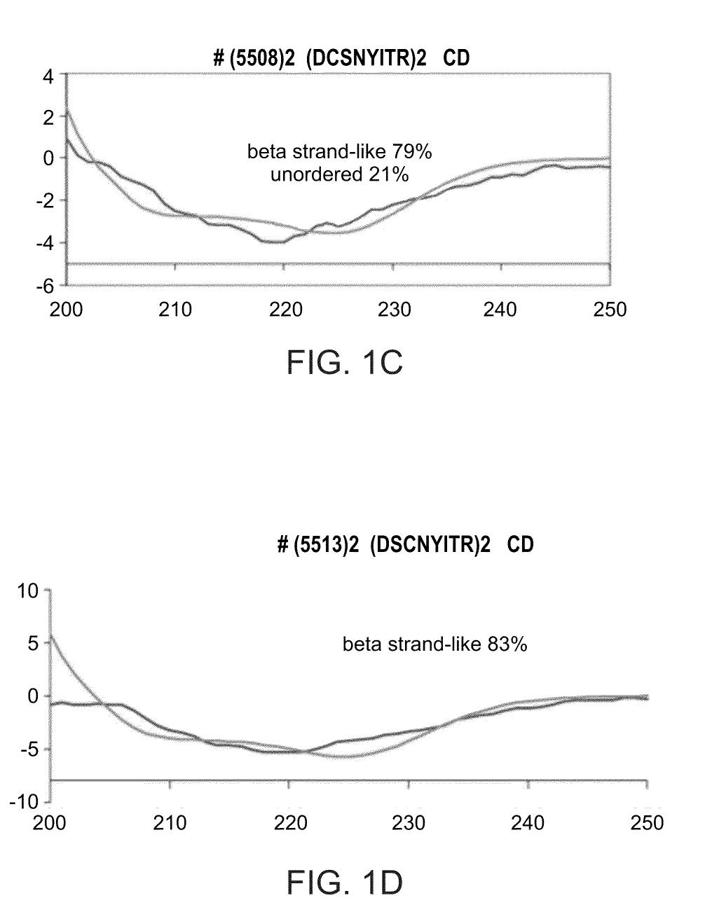

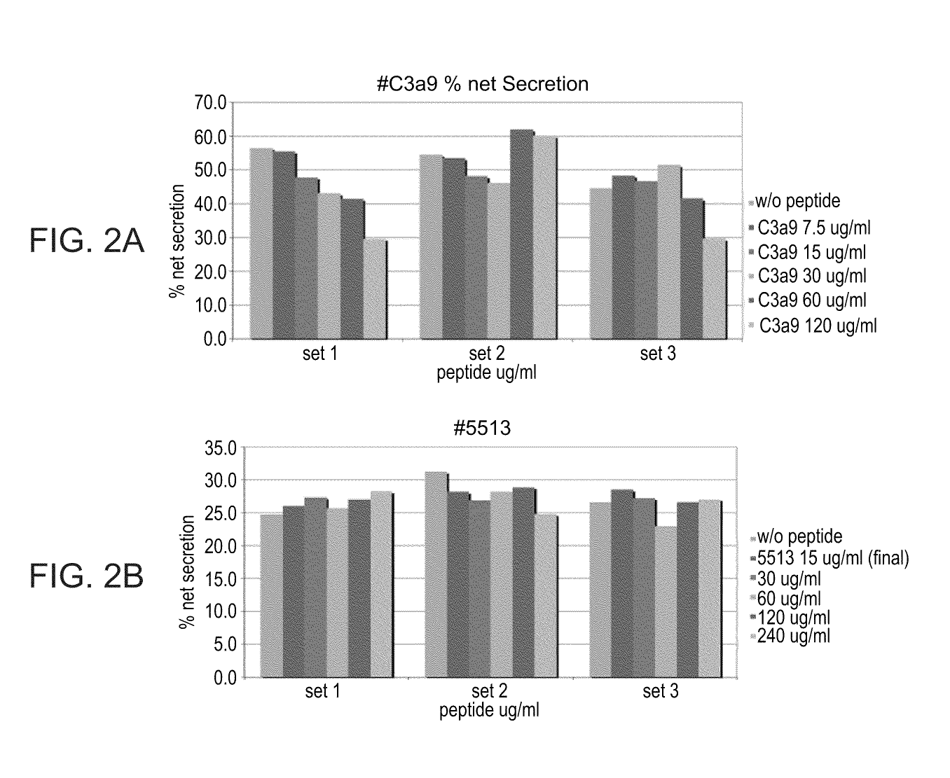

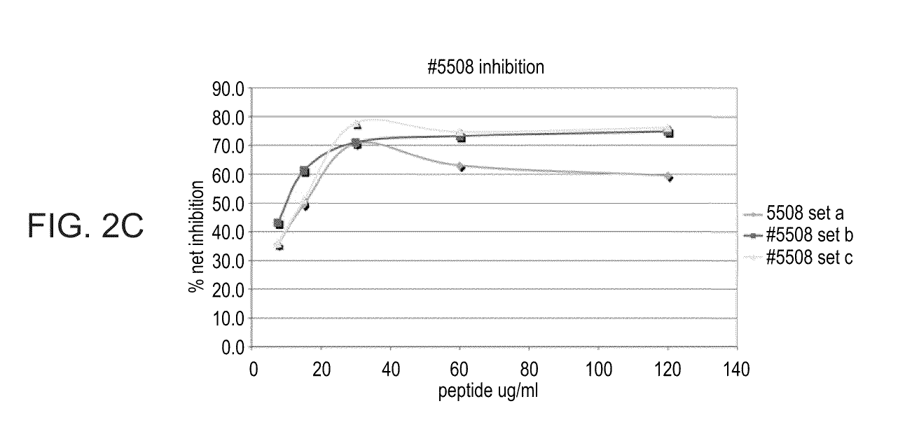

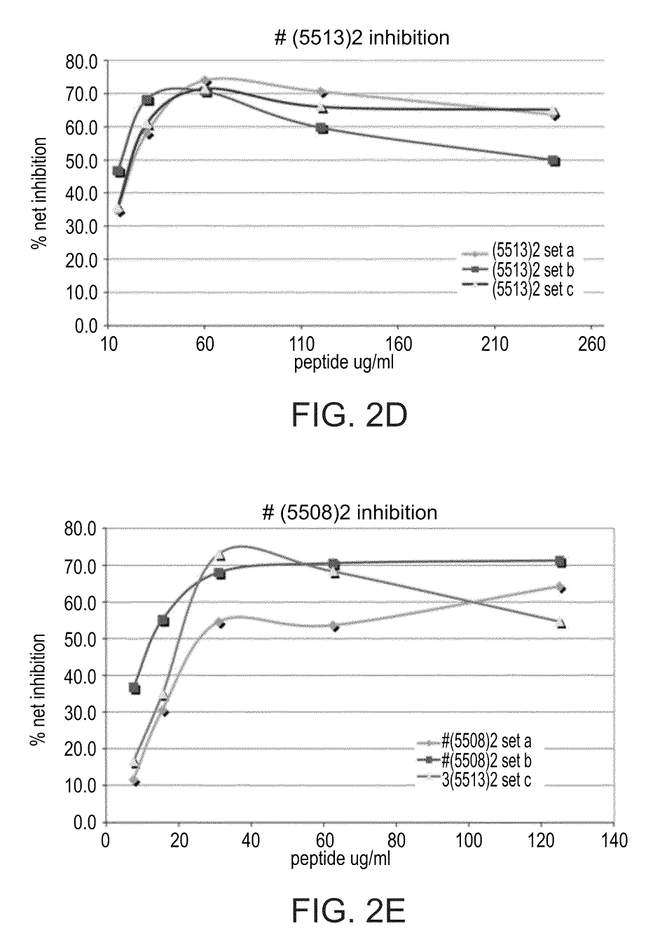

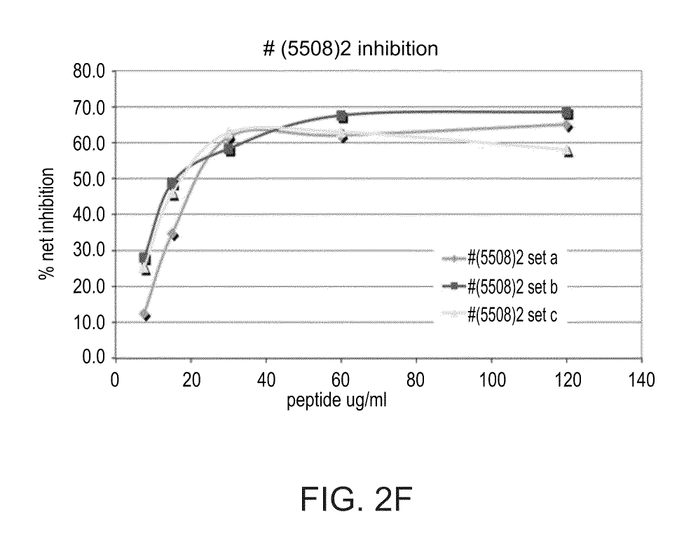

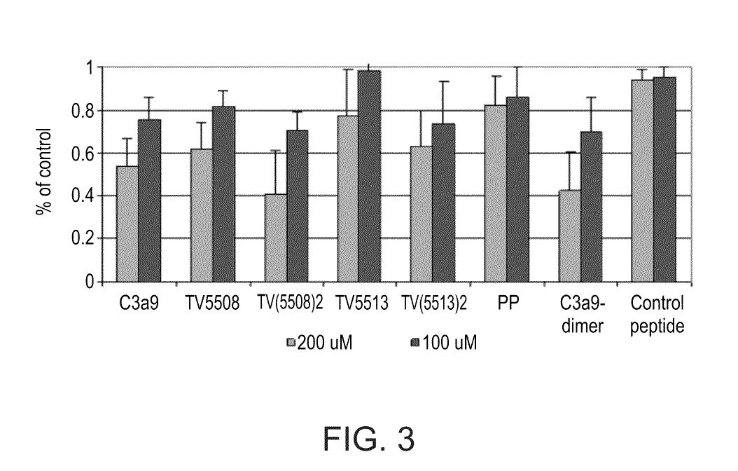

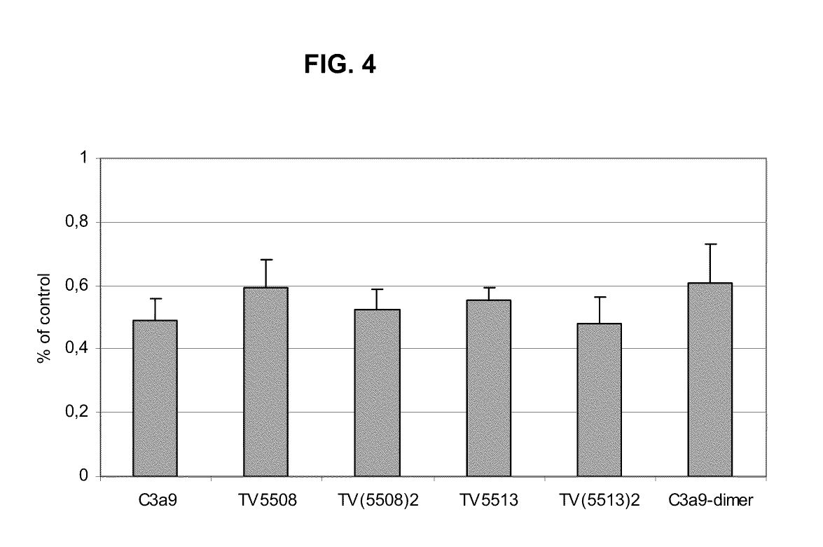

As illustrated in the Examples section below, the present inventors showed that both these dimers are soluble in water and exhibit circular dichroism (Table 3 and FIGS. 1A-D). In addition, the present inventors showed that both dimers prevent degranulation of mast cells and human basophils, as measured by monitoring activity of released β-hexoseaminidase from a rat mucosal-type mast cell line (FIGS. 2A-F) and analysis of expression of CD63, a granular membrane protein on the surface of human basophils by flow cytometry (FIG. 3). Finally, the present inventors demonstrated that both dimers prevent in vivo histamine release in mice following passive systemic anaphylaxis (FIG. 4).

These findings demonstrate an improved pharmacological profile of dimeric peptides derived from and corresponding partially to the amino acid sequence of positions 55-64 of human complement peptide C3a, and accordingly, demonstrate an improved effect of these dimeric peptides in treating allergic disorders.

Thus, according to one aspect of the present invention there is provided a peptide composition comprising a dimeric peptide, said dimeric peptide comprising two peptide monomers, each independently comprising the amino acid sequence:

Asp-X1-X2-Asn-Tyr-Ile-Thr-X3

wherein:
X1 is selected from the group consisting of Cys and a Cys derivative;
X2 is selected from the group consisting of Cys and a Cys derivative; and
X3 is selected from the group consisting of Arg and Glu-Leu-Arg, provided that at least one of X1 and X2 is Cys,

wherein a mol percentage of said dimeric peptide in the composition is at least 50 mol percents.

As used herein, the phrase “peptide composition” describes a product of a peptide synthesis. As is well known in the art, a product of a peptide synthesis typically comprises peptide(s) having a desired sequence, as defined herein, contaminated by peptides having an undesired sequence, as defined herein.

In the context of embodiments of the present invention, the phrase “peptide composition” describes a product of a peptide synthesis in which peptide monomers are subjected to reaction conditions that allow or promote bond formation between two amino acids.

The phrase “dimeric peptide” refers to a peptide having two peptide monomers associated covalently by covalent interactions. Thus, dimeric peptides, as used herein, include at least one intermolecular covalent bond. Dimeric peptides, however, can further exhibit non-covalent bonds, both intramolecular and intermolecular non-covalent bonds.

In exemplary embodiments, a peptide composition is a product or a peptide synthesis in which monomeric peptides are subjected to reaction conditions that allow or promote formation of one or more disulfide bridge between two cysteine residues.

The phrase “peptide composition” encompasses both, a product of a peptide synthesis isolated from the reaction medium in which the peptide was prepared, and such a product contained within the reaction medium in which the peptide was prepared. This phrase further encompasses a product of a peptide synthesis which is isolated from the reaction medium in which the peptide was prepared and is further purified, such that a peptide having the desired amino acid sequence is separated from peptides having an undesired sequence.

In the context of the present embodiments, the phrase “a peptide having a desired sequence” describes a dimeric peptide that comprises two peptide monomers which are bound to one another via pre-determined intermolecular bonds (e.g., one or more pre-determined disulfide bridges) between pre-determined amino acids in each peptide monomer.

The phrase “a peptide having an undesired sequence” describes any of the following: (i) peptide monomers which have a secondary structure other than the monomeric peptides used for synthesizing the peptide product; (ii) dimeric peptides that comprises two peptide monomers which are bound to one another via bonds between one or more amino acids in one peptide monomer and one or more amino acids in the other peptide monomer, and which have a different primary structure as compared with a dimeric peptide having a desired sequence, as defined herein; and (iii) oligomeric peptides, comprising 3 or more peptides that are bound to one another.

Thus, the phrase “a peptide having an undesired sequence” in the context of embodiments of the invention, therefore encompasses monomeric and/or oligomeric peptides, and dimeric peptides that differ from dimeric peptides having a desired sequence by the position, chemical structure and/or number of amino acid residues that participate in the intermolecular bond(s) that links two peptide monomers in the dimeric peptide.

A peptide composition, as described herein, is typically in a form of a solid or a solution. Solid compositions can be, for example, powdered or lyophilized. The composition can further be bound to a solid support.

Herein throughout, the phrase “peptide monomer” is used to describe a peptide monomer that forms a part of a dimeric peptide, unless otherwise indicated. The phrase “monomeric peptide” is used to describe a peptide in a monomeric form thereof (which does not form a part of a dimeric or oligomeric peptide), unless otherwise indicated.

According to embodiments of the invention, the peptide composition comprises a dimeric peptide formed from two peptide monomers, each peptide monomer being derived from and corresponding partially to the sequence of amino acids 55-64 of human complement C3a, and each comprising at least one cysteine residue, such that the dimeric peptide comprises one or more intermolecular disulfide bridges formed between two cysteine residues.

Thus, in some embodiments of the invention, each of the peptide monomers comprises an amino acid sequence as described herein, in which at least one of X1 and X2 is a cysteine residue, such that the peptide monomers are covalently linked to one another via a disulfide bridge formed between a cysteine residue in one peptide monomer and a cysteine residue in another peptide monomer.

In some embodiments, the peptide monomers forming the dimeric peptide are covalently linked to one another via a single disulfide bridge, as further detailed hereinbelow.

In some embodiments, the peptide monomers forming the dimeric peptide are covalently linked to one another via two disulfide bridges, as further detailed hereinbelow.

Hereinthroughout, the phrases “disulfide bridge” and “disulfide bond” are used interchangeably, and describe a —S—S— bond.

It is to be noted that the disulfide bridges referred to herein relate to the part of the dimeric peptide that is formed by the portions of the peptide monomers that have the amino acid sequence described herein. Thus, in cases where the one or both peptide monomers comprises additional amino acids, the dimeric peptides may have additional intermolecular bonds, including additional disulfide bridges, linking two amino acids other than those forming the amino acid sequence described herein.

As further detailed hereinbelow, the peptide composition described herein was designed and successfully prepared via a directed synthesis, for obtaining a desired dimeric peptide in which one or two intermolecular disulfide bridge is formed between the two peptide monomers, at a predetermined position (namely, between two predetermined cysteines).

The peptide compositions described herein is therefore such that a majority of the molecules in the composition are a dimeric peptide of a desired, pre-determined chemical structure.

Thus, in some embodiments, the peptide composition described herein comprises at least 50 mol percents of the dimeric peptide, at least 60 mol percents of the dimeric peptide, at least 70 mol percents of the dimeric peptide, at least 80 mol percents of the dimeric peptide, at least 90 mol percents of the dimeric peptide, at least 95 mol percents of the dimeric peptide, at least 98 mol percents of the dimeric peptide, at least 99 mol percents of the dimeric peptide, at least 99.1 mol percents, at least 99.2 mol percents, at least 99.3 mol percents, at least 99.4 mol percents, at least 99.5 mol percents, at least 99.6 mol percents, at least 99.7 mol percents, at least 99.8 mol percents, at least 99.9 mol percents, at least 99.95 mol percents, at least 99.96 mol percents, at least 99.97 mol percents, at least 99.98 mol percents, at least 99.99 mol percents, and even consists of the dimeric peptide, such that it includes 100 mol percents of the dimeric peptide (e.g., an isolated dimeric peptide).

By “mol percents” it is meant the partial number of molecules of the dimeric peptide out of the total number of molecules in the peptide composition.

As is well recognized by those skilled in the art, the synthesis of peptides comprising disulphide bridges is challenging since it is difficult to ensure that the correct cysteine residues combine to form the desired disulphide bridges.

Thus, for example, in peptide compositions as described herein, in which one or both of the peptide monomers comprise two cysteine residues (as X1 and X2), dimeric peptides can be formed with each of the cysteine residues, as exemplified hereinafter, so as to form a mixture of dimeric species. In addition to the mixture of such dimeric species, oligomeric peptides can be formed. Furthermore, peptide monomers in which the two cysteines are combined intramolecularly can be formed.

However, in some embodiments, the peptide compositions described herein can be such that include dimeric peptides in which selected cysteine residues combine to form a disulfide bridge.

In some embodiments, the peptide compositions described herein thus consist of a single (homo or hetero) dimeric species, as defined herein, in which a selected cysteine residue in each peptide monomer (X1 or X2, as described herein) participates in the formed disulfide bridge(s).

In some embodiments of the invention, a mol percentage of a oligomeric peptide, a monomeric peptide and/or a dimeric peptide other than the dimeric peptide described herein is lower than 1 mol percent, and can be lower than 0.5 mol percent, lower than 0.1 mol percent, lower than 0.05 mol percent, lower than 0.01 mol percent, and even lower.

In some embodiments, the peptide composition is devoid of oligomeric peptides, monomeric peptides and dimeric peptides other than the dimeric peptides described herein.

The phrase “oligomeric peptide” as used herein, describes a peptide formed from three or more peptide monomers that are covalently linked therebetween, optionally via disulfide bridges. The phrase “dimeric peptide other than said dimeric peptide” describes a dimeric peptide which comprises intermolecular bonds other than those described herein. Examples of such other dimeric peptide include, but are not limited to, dimeric peptides that differ from one another by the number and/or position of the disulfide bonds, a dimeric peptide which includes bonds other than disulfide bridges, and any other dimeric peptide not encompassed by embodiments of the invention.

In some embodiments, the peptide composition is such that the amino acid sequence of each of the two peptide monomers in the dimeric peptide are the same, forming a homodimeric peptide. In cases where one of or both monomeric peptides that combine to form a homodimeric peptide comprise two cysteine residues, several homodimeric species, which differ from one another by the cysteine residues that participate in the disulfide bridge, can be formed.

In some embodiments, the peptide composition is such that the amino acid sequence of each of the two peptide monomers in the dimeric peptide is different, forming a heterodimeric peptide. Heterodimeric peptides can also include heterodimeric species, as detailed hereinafter.

In some embodiments, in one or both peptide monomers, each of X1 and X2 is a cysteine residue.

In some embodiments, a peptide monomer in which each of X1 and X2 is a cysteine residue comprises the amino acid sequence Asp-Cys-Cys-Asn-Tyr-Ile-Thr-Arg, as set forth in SEQ ID NO:3, also referred to herein as C3a9.

In some embodiments, a peptide monomer in which each of X1 and X2 is a cysteine residue comprises the amino acid sequence Asp-Cys-Cys-Asn-Tyr-Ile-Thr-Glu-Leu-Arg, as set forth in SEQ ID NO:2, also referred to as C3a7.

In embodiments where in one or both peptide monomers, each of X1 and X2 is a cysteine residue, the peptide composition can comprise dimeric peptides in which intermolecular disulfide bridges are formed between any combinations of the two cysteine residues, as is further detailed hereinbelow.

Dimeric peptides that are formed from two peptide monomers in which each of X1 and X2 is a cysteine residue, and which have the same amino acid sequences, may form different homodimeric species.

By “homodimeric species” it is meant that a dimeric peptide comprises two monomeric peptides that have identical amino acid sequences, and one or two disulfide bridges, each linking t a cysteine residue at one position of one monomer and a cysteine residue at one position of the other monomer. That is, a homodimeric species may have a disulfide bridge linking X1 in one monomer to X1 in another monomer; a disulfide bridge linking X1 in one monomer to X2 in another monomer; a disulfide bridge linking X2 in one monomer to X2 in another monomer; two disulfide bridges linking X1 in one monomer to X1 in another monomer and X2 in one monomer to X2 in another monomer; and two disulfide bridges linking X1 in one monomer to X2 in another monomer and X2 in one monomer to X1 in another monomer.

For example, dimeric peptides formed from two peptide monomers having SEQ ID NO:3 (C3a9), can have the following structures:

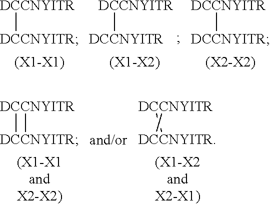

These structures represent exemplary different homodimeric species.

In some embodiments, the dimeric peptide comprises a mixture of homodimeric species.

In some embodiments, the dimeric peptide comprises a single homodimeric species.

In some embodiments, in at least one of the peptide monomers, one of X1 and X2 is a cysteine derivative.

As used herein, a “cysteine derivative” or “a Cys derivative” describes a structural analog of cysteine, which does not comprise a free thiol group.

According to some embodiments of the invention, cysteine derivatives have the following structure:

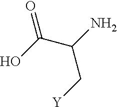

wherein Y is other than thiol.

As used herein, the term “thiol” describes as —SH group.

Thus, in some embodiments, the Cys derivative is devoid of a free thiol group.

In some embodiments, Y is a —OH group, such that the cysteine derivative is a Serine residue.

Exemplary peptide monomers in which the cysteine derivative is Ser have, as non-limiting example, the amino acid sequence Asp-Cys-Ser-Asn-Tyr-Ile-Thr-Arg as set forth in SEQ ID NO:4 (also referred to herein as Monomer A or 5508); or the amino acid sequence Asp-Ser-Cys-Asn-Tyr-Ile-Thr-Arg as set forth in SEQ ID NO:5 (also referred to herein as Monomer B, or 5513).

In some embodiments, Y is a Cysteine protecting group, such that the Cys derivative is a protected Cys residue.

The phrase “protected Cys residue” describes a cysteine residue in which the free thiol group at the side chain is derivatized by a chemical group that can be removed, so as to regenerate, under certain conditions, a free thiol group. Such chemical groups are known as cysteine protecting groups.

Exemplary cysteine protecting groups include, but are not limited to, benzyl, MBzl, 4-methoxybenzyl, trityl, methoxytrityl, tBu, t-butylthiol, acetyl, 3-nitro-2-pyridinesulphenyl and Acm [see, for example, Barany and Merrifield in The Peptides” Vol. 2, Ed. Gross and Minehoffer, Academic Press, pp. 233-240 (1980)].

In some embodiments, Y is a chemical group other then thiol, and can be, as non-limiting examples, halide, amide, nitro, cyano, sulfonamide, carbonyl, thiocarbonyl, carboxylate, carbamate, thiocarboxylate, phosphonate, thiophosphonate, phosphinyl, alkyl, haloalkyl, cycloalkyl, aryl, heteroalicyclic, heteroaryl, acyl-halide, sulfonate, sulfoxide, thiosulfate, sulfate, alkoxy, aryloxy, thioalkoxy, thioaryloxy, isocyanate, thiocarbamate, urea, thiourea, guanyl, or guanidine, as these terms are defined herein.

In some embodiments, the Cys derivative is an amino acid or an analog thereof which do not affect properties such as water solubility, hydrophilicity, and the desired activity, of the peptide monomer and the dimer formed therefrom. An exemplary such amino acid is Thr. Exemplary such peptide monomers have an amino acid sequence as set forth is SEQ ID NO:6 and SEQ ID NO:7.

In embodiments where one peptide monomer has the amino acid sequence as described herein in which both X1 and X2 are cysteine and the other monomer has the amino acid sequence as described herein in which one of X1 and X2 is cysteine and the other is a cysteine derivative, as described herein, the dimeric peptide is a heterodimer. In such a heterodimer, two heterodimer species can be formed, by the formation of a disulfide bridge between the single cysteine residue in one monomer and one of the two cysteine residues in the other monomer.

For example, in cases where one monomer has the amino acid sequence as set forth in SEQ ID NO:3 (C3a9), and one monomer has the amino acid sequence as set forth in SEQ ID NO:4 (Monomer A), the following heterodimer species can be formed:

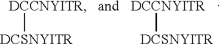

In cases where one monomer has the amino acid sequence as set forth in SEQ ID NO:3 (C3a9), and one monomer has the amino acid sequence as set forth in SEQ ID NO:5 (Monomer B), the following heterodimer species can be formed:

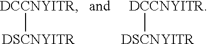

In some embodiments, in each of the peptide monomers forming the dimeric peptide, one of X1 and X2 is cysteine and the other is a cysteine residue, as described herein.

Such dimeric peptides can be heterodimeric peptides, where the peptide monomers differ from one another by the position of the cysteine residue, and/or by the nature of the cysteine derivative. Such dimeric peptides can alternatively be homodimeric.

An exemplary such heterodimeric peptide is:

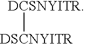

Exemplary homodimeric peptides include:

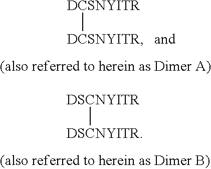

In some embodiments, the dimeric peptides described herein consist of the amino acid sequence set forth hereinabove, as exemplified hereinabove.

Alternatively, the dimeric peptides may comprise the amino acid sequence set forth hereinabove, and may further comprise additional amino acid residues.

Accordingly, in each of the exemplary structures set forth hereinabove for dimeric peptides, additional amino acid residues can be included in one or both peptide monomers. Such additional amino acid residues may render the exemplary homodimeric peptides and homodimeric species, heterodimeric peptides and heterodimeric species, respectively, if the additional amino acid residues are not identical in both peptide monomers.

In some embodiments, the dimeric peptides described herein are characterized by an ordered beta sheet structure, as is further detailed hereinunder.

As demonstrated in the Examples section that follows, and in FIGS. 1A-D, the ordered beta sheet structure was determined by circular dichroism measurements.

The phrase “circular dischroism”, as used herein and known in the art, describes the differential absorption left-handed and right-handed circularly polarized light.

As further demonstrated in the Examples section that follows, and is detailed hereinunder, the peptide compositions described herein exhibit anti-allergic activity.

As would be appreciated by any person skilled in the art, the anti-allergic activity exhibited by such dimeric peptides, and particularly an improved anti-allergic activity as compared with the previously described monomeric peptides, is non-trivial, since in the formed dimeric peptides, at least one reactive amino acid (namely, an amino acid that forms a part of an active site of the previously described monomeric peptide) is chemically modified, and yet, the biological activity is maintained and even improved.

As would further be appreciated, the chemical modification relates to (i) the formation of a disulfide bridge (oxidation of a Cys residue); and (ii) the replacement of Cys by a Cys derivative.

Further according to an aspect of embodiments of the present invention there is provided a process of preparing the peptide composition as described herein. The process is effected by reacting two monomeric peptides, each independently comprising the amino acid sequence:

Asp-X1-X2-Asn-Tyr-Ile-Thr-X3

wherein:
X1 is selected from the group consisting of Cys and a Cys derivative;
X2 is selected from the group consisting of Cys and a Cys derivative; and
X3 is selected from the group consisting of Arg and Glu-Leu-Arg,
provided that at least one of X1 and X2 is Cys, as described herein, in the presence of an oxidizing agent.

The oxidizing agent is selected so as to promote formation of a disulfide bridge between two cysteine residues.

Exemplary oxidizing agents include iodine, preferably in the form of an aqueous solution of iodine, and any other oxidizing agents known in the art as promoting formation of a disulfide bridge between 2 cysteine residues.

In some embodiments, reacting the monomeric peptides is performed under conditions that favor intermolecular interactions between the peptide monomers.

Thus, the peptide compositions described herein are prepared under conditions that do not favor, and even do not enable, an intramolecular reaction, even in the presence of an oxidizing agent.

As known in the art, intramolecular reactions are often favored when a reaction mixture is diluted to such an extent where intermolecular interactions between the reacting molecules are not energetically favored, whereby intramolecular interactions of one or more reactants (if feasible) are energetically favored. Accordingly, in some embodiments, reacting the monomeric peptides in the presence of an oxidizing agent is performed in a solution, namely, in the presence of a solvent, whereby the concentration of each of the monomeric peptides in the solution is such that intramolecular interactions are not favored, and intermolecular interactions are favored.

Thus, in some embodiments, a concentration of each of said monomeric peptides in said solvent is at least 20 μM, at least 50 μM, at least 100 μM, at least 200 μM, at least 300 μM, at least 400 μM, at least 500 μM, and can be even 1 mM.

It is to be noted, however, that a concentration of a monomeric peptide in the solvent at which intermolecular interactions are favored depends also on the type, 22 reactivity and concentration of the oxidizing agent, on the chemical structure of the peptide, and/or on the type of solvent.

As noted hereinabove, the synthesis of peptides comprising disulphide bridges is challenging since it is difficult to ensure that the correct cysteine residues combine to form the desired disulphide bridges.

As further known in the art, in a concentrated reaction mixture, intermolecular reactions are favored, yet, such conditions further favor the formation of numerous species of dimeric peptides and even oligomeric peptides.

In order to obtain defined dimeric peptides, in which the cysteine residues that combine to form a disulfide bridge are pre-determined, the present inventors utilized various methodologies.

In one methodology, monomeric peptides comprising an amino acid sequence as set forth hereinabove, in which in one or both monomeric peptide(s) at least one of X1 and X2 is a Cys derivative, as defined herein, were used.

A Cys derivative, as described herein, is devoid of a free thiol group and hence do not participate in the formation of a disulfide bridge. Monomeric peptides which comprise a Cys derivative include only a single Cys residue that participate in the formation of a disulfide bridge, and the formation of numerous dimeric species is avoided or at least reduced.

Thus, in some embodiments, at least one of the monomeric peptides used for forming the dimeric peptide comprises a Ser residue as X1 or X2, thus forming dimeric peptide with a disulfide bond only between the remaining Cys residues.

In some embodiments, two monomeric peptides, each comprising a Ser residue as X1 or X2 were used, thus forming dimeric peptide with a disulfide bond only between the remaining two Cys residues.

In another methodology, monomeric peptides which comprise two Cys residues are selectively protected, so as to obtain one Cys residue and one protected Cys residue, as X1 and X2 in the amino acid sequence described herein.

In another methodology, peptide monomers which comprise two cysteine residues are protected so as obtain two protected Cys residues, and are thereafter subjected to selective deprotection, so as to obtain one Cys residue and one protected Cys residue, as X1 and X2.

The latter methodology can be further used to selectively form dimeric peptides with two disulfide bridges. Thus, upon obtaining two protected Cys residues, the monomeric peptides are subjected to sequential deprotection and oxidation procedures, under such conditions that a first protected Cys residue is deprotected in each monomeric peptide, a first oxidation is thereafter effected so as to form a first disulfide bridge between pre-selected Cys residues, and then a second Cys residue is deprotected, under conditions that do not allow reduction of the first disulfide bridge, and a second oxidation is thereafter effected, so as to form a second disulfide bridge.

Using any of these methodologies thus enables to obtain dimeric peptides in which one or two disulfide bridge(s) is formed between pre-selected cysteine residues.

It is to be noted that a process as described herein can be utilized also for obtaining pre-selected homodimeric or heterodimeric species. For example, a solution of a monomeric peptide which comprises two cysteine residues can be treated, as described hereinabove, such that one of the cysteine residues is selectively protected, and is then subjected to oxidation, as described herein. Alternatively, two solutions of a monomeric peptide which comprises two cysteine residues are prepared, and each solution is treated differently, so as to obtain a different cysteine residue that is selectively protected, and in then subjected to oxidation. Further alternatively, a solution of a monomeric peptide which comprises two cysteine residues can be treated, as described hereinabove, such that one of the cysteine residues is selectively protected, and this solution is then reacted with a peptide monomer is which one of X1 and X2 is Ser.

It is to be noted that in cases where a protected Cys residue is utilized, deprotection can be performed once the dimeric peptide is obtained, so as to regenerate a Cys residue.

The monomeric peptides utilized for forming the dimeric peptides described herein can be synthesized using methods well known in the art, including chemical synthesis and recombinant DNA technology. Synthesis may be performed by solid phase peptide synthesis described by Merrifield (see J. Am. Chem. Soc., 85:2149, 1964). Alternatively, the peptide monomers can be synthesized using standard solution methods (see, for example, Bodanszky, M., Principles of Peptide Synthesis, Springer-Verlag, 1984). Preferably, the peptides of the invention are synthesized by solid phase peptide synthesis as exemplified herein below (Example 1).

Cysteine protecting groups and methods of utilizing same for forming protected Cysteines are also known in the art. Any known methodology for forming a protected Cys residue and for deprotecting a protected Cys residue, either selectively or non-selectively, is contemplated herein. Particularly useful are methods for deprotecting a protected Cys residue under conditions that do not enable reduction of a disulfide bridge.

Exemplary methodologies are described in U.S. Pat. No. 6,906,171, which is incorporated by reference as if fully set forth herein.

The term “alkyl”, as used herein, describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Representative examples are piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted.

The terms “halide” and “halo” describes fluorine, chlorine, bromine or iodine.

The term “haloalkyl” describes an alkyl group as defined above, further substituted by one or more halide.

The term “sulfate” describes a —O—S(═O)₂—OR′ group, where R′ can be hydrogen, alkyl, cycloalkyl or aryl, as defined herein.

The term “thiosulfate” describes a —O—S(═S)(═O)—OR′ group, where R′ is as defined hereinabove.

The term “sulfoxide” or “sulfinyl” describes a —S(═O)R′ group, where R′ is as defined hereinabove.

The term “sulfonate” describes a —S(═O)2—R′ group, where R′ is as defined herein.

The term “sulfonamide” describes a —S(═O)₂—NR′R″ group or a R′S(═O)₂—NR″— group, where R′ is as defined herein and R″ is as defined herein for R′.

The term “phosphonate” describes a —P(═O)(OR′)(OR″) group, with R′ and R″ as defined herein.

The term “thiophosphonate” describes a —P(═S)(OR′)(OR″) group, with R′ and R″ as defined herein.

The term “phosphinyl” describes a —PR′R″ end group or a —PR′— linking group, as these phrases are defined hereinabove, with R′ and R″ as defined hereinabove.

The term “carbonyl” or “carbonate” as used herein, describes a —C(═O)—R′ group, with R′ as defined herein.

The term “thiocarbonyl ” as used herein, describes a —C(═S)—R′ group, with R′ as defined herein.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thioalkoxy” describes both a —S-alkyl group, and a —S-cycloalkyl group, as defined herein.

The term “thioaryloxy” describes both a —S-aryl and a —S-heteroaryl group, as defined herein.

The term “cyano” describes a —C≡N group.

The term “isocyanate” describes an —N═C═O group.

The term “nitro” describes an —NO₂ group.

The term “acyl halide” describes a —(C═O)R″″ group wherein R″″ is halide, as defined hereinabove.

The term “carboxylate” describes a —C(═O)—OR′ group or a —OC(═O)R′ group, where R′ is as defined herein.

The term “thiocarboxylate” describes a —C(═S)—OR′ group or a —OC(═S)R′ group, where R′ is as defined herein.

The term “carbamate” describes an R″OC(═O)—NR′— group or an —OC(═O)—NR′R″ group, with R′ and R″ as defined herein.

The term “thiocarbamate” describes a —OC(═S)—NR′R″ group or a R″OC(═S)NR′— group, with R′ and R″ as defined herein.

The term “urea”, which is also referred to herein as “ureido”, describes a —NR′C(═O)—NR″R′″ group or a —NR′C(═O)—NR″— linking group, where R′ and R″ are as defined herein and R′″ is as defined herein for R′ and R″.

The term “thiourea”, which is also referred to herein as “thioureido”, describes a —NR′—C(═S)—NR″R′″ group, with R′, R″ and R′″ as defined herein.

The term “amide” describes a R′C(═O)—NR″— group or a R′R″NC(═N)— group, where R′ and R″ are as defined herein.

The term “guanidine” describes a —R′NC(═N)—NR″R′″ group, where R′, R″ and R′″ are as defined herein.

The present invention further encompasses pharmaceutically acceptable salts, prodrugs, solvates and hydrates of any of the monomeric, dimeric and multimeric peptides described herein.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

Exemplary salts include salts of carboxyl groups and acid addition salts of amino groups of the multimeric, dimeric or monomeric peptide. Salts of carboxyl groups can be formed by means known in the art and include inorganic salts, for example aluminum, ammonium, calcium, copper, ferric, ferrous, lithium, magnesium, manganic, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Acid addition salts include, for example, salts with mineral acids such as, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

As used herein, the term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. The prodrug may also have improved solubility as compared with the parent drug in pharmaceutical compositions. Prodrugs are also often used to achieve a sustained release of the active compound in vivo.

The term “solvate” refers to a complex of variable stoichiometry (e.g., di-, tri-, tetra-, penta-, hexa-, and so on), which is formed by a solute (the peptide) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

The term “peptide” as used herein refers to a polymer of natural or synthetic amino acids, encompassing native peptides (either degradation products, synthetically synthesized polypeptides or recombinant polypeptides) and peptidomimetics (typically, synthetically synthesized peptides), as well as peptoids and semipeptoids which are peptide analogs, which may have, for example, modifications rendering the peptides even more stable while in a body or more capable of penetrating into cells.

Such modifications include, but are not limited to N terminus modification, C terminus modification, polypeptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within any of the monomeric, dimeric or oligomeric peptide described herein may be substituted, for example, by N-methylated bonds (—N(CH₃)−CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), polypeptide derivatives (—N(R)—CH₂—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the polypeptides of the present invention may also include one or more modified amino acids or one or more non-amino acid monomers (e.g. fatty acids, complex carbohydrates etc), other than those described hereinabove.

As used herein in the specification and in the claims section below the term “amino acid” or “amino acids” is understood to include the 20 naturally occurring amino acids;

those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers).

Tables 1 and 2 below list naturally occurring amino acids (Table 1) and non-conventional or modified amino acids (Table 2) which can be used with the present invention.

TABLE 1

| Amino Acid | Three-Letter Abbreviation | One-letter Symbol |
|---|---|---|
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α ethylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α thylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α ethylhistidine | Mhis | L-α-methylhomophenylalanine | Mhphe |
| L-α thylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α ethylvaline | Mtrp | L-α-methyltyrosine | Mtyr |
| L-α-methylleucine | Mval | L-N-methylhomophenylalanine | Nmhphe |
| | nbhm | | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| hylamino)cyclopropane | | | |

The N and C termini of the peptides of the present invention may be protected by function groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

These moieties can be cleaved in vivo, either by hydrolysis or enzymatically, inside the cell. Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described herein for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester.

Examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3—O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH3)2, —NH(ethyl), —N(ethyl)₂, —N(methyl)(ethyl), —NH(benzyl), —N(C1-C4 alkyl)(benzyl), —NH(phenyl), —N(C1-C4 alkyl)(phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

The peptides of the present embodiments may also comprise non-amino acid moieties, such as for example, hydrophobic moieties (various linear, branched, cyclic, polycyclic or heterocyclic hydrocarbons and hydrocarbon derivatives) attached to the peptides; various protecting groups, especially where the compound is linear, which are attached to the compound's terminals to decrease degradation. Chemical (non-amino acid) groups present in the compound may be included in order to improve various physiological properties such; decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity and the like.

According to one embodiment, the peptides of the present embodiments are attached to a sustained-release enhancing agent. Exemplary sustained-release enhancing agents include, but are not limited to hyaluronic acid (HA), alginic acid (AA), polyhydroxyethyl methacrylate (Poly-HEMA), polyethylene glycol (PEG), glyme and polyisopropylacrylamide.

Attaching the amino acid sequence component of the peptides of the invention to other non-amino acid agents may be by covalent linking, by non-covalent complexion, for example, by complexion to a hydrophobic polymer, which can be degraded or cleaved producing a compound capable of sustained release; by entrapping the amino acid part of the peptide in liposomes or micelles to produce the final peptide of the invention. The association may be by the entrapment of the amino acid sequence within the other component (liposome, micelle) or the impregnation of the amino acid sequence within a polymer to produce the final peptide of the invention.

The compounds described herein may be linear or cyclic (cyclization may improve stability). Cyclization may take place by any means known in the art. Where the compound is composed predominantly of amino acids, cyclization may be via N- to C-terminal, N-terminal to side chain and N-terminal to backbone, C-terminal to side chain, C-terminal to backbone, side chain to backbone and side chain to side chain, as well as backbone to backbone cyclization. Cyclization of the peptide may also take place through non-amino acid organic moieties comprised in the peptide.

The peptides of the present invention can be biochemically synthesized such as by using standard solid phase techniques. These methods include exclusive solid phase synthesis, partial solid phase synthesis methods, fragment condensation, classical solution synthesis. Solid phase peptide synthesis procedures are well known in the art and further described by John Morrow Stewart and Janis Dillaha Young, Solid Phase Polypeptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

Recombinant techniques may also be used to generate the peptides of the present invention. To produce a peptide of the present invention using recombinant technology, a polynucleotide encoding the peptide of the present invention is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

In addition to being synthesizable in host cells, the polypeptide of the present invention can also be synthesized using in vitro expression systems. These methods are well known in the art and the components of the system are commercially available.

According to this aspect of the present invention, the dimeric peptides comprise anti-allergic activity. Such activities may be assayed using various known in vitro assay systems and in vivo animal models such as those described in the Examples section below.

Since the peptides of the present invention are constrained into dimers, typically they may be characterized by a well-ordered structure, such as for example a B sheet structure.

Since the dimeric peptides of the present invention comprise anti-allergic properties they may be used to treat allergic conditions.

Thus, according to another aspect of the present invention there is provided a method of treating an allergic disorder. The method comprises administering to a subject in need thereof a therapeutically effective amount of the peptide compositions of the present invention.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Contemplated subjects that may be treated with the dimeric peptides of the present invention include mammals, such as human beings.

Exemplary allergic disorders that may be treated according to this aspect of the present invention are those mediated by a cell type selected from the group consisting of mucosal-type mast cells, serosal-type mast cells and/or basophils, without inducing an anaphylatoxic effect. In some embodiments, the disorder is an allergic disorder resulting from an IgE- or IgG-mediated (Type I or Type III) hypersensitivity and/or FcεRI- or FcγR-induced secretory response.

Examples of allergic diseases that can be treated by the dimeric peptides of the invention include, but are not limited to, allergic rhinitis, including seasonal rhinitis and sinusitis; pulmonary diseases, such as bronchial asthma; allergic dermatosis, such as urticaria, angioedema, eczema, atopic dermatitis, and contact dermatitis; allergic conjuctivitis; gastrointestinal allergies such as those caused by food or drugs; cramping; nausea; vomiting; diarrhea; irritable bowel disease; and ophthalmic allergies such as uveitis; cheilitis; vulvitis; and anaphylaxis. The present invention is also useful in alleviating or treating the symptoms induced by exposure to toxins, including bee venom toxins and the like. In a certain embodiment, the allergic disorder is asthma.

For the treatment of hay fever, for example, the peptides may be formulated in the form of spray, aerosol or drops that can be appropriate for administration to subjects in need to prevent the development of allergy in the pollen-season. Moreover, it is well known that the bronchial mucosal surface is the first contact site for inhaled allergens and, consequently, the response of mast cells to the inhibitory peptides of the invention administered as spray may be very effective.

Allergic disorders associated with serosal mast cell activation include, but are not limited to, Type I or Type III immediate hypersensitivity reactions such as gastrointestinal allergies, cramping, nausea, vomiting, and diarrhea.

Further according to an aspect of embodiments of the invention there is provided a use of dimeric peptides described herein in the manufacture of a medicament for treating an allergic disorder, as described herein.

In any of the methods and uses described herein the dimeric peptides of the present embodiments may be utilized per se or as part of a pharmaceutical composition.

Hence, according to another aspect of embodiments of the invention, there is provided a pharmaceutical composition which comprises a dimeric peptide as described herein and a pharmaceutically acceptable carrier.

The phrase “pharmaceutical composition”, as used herein refers to a preparation of one or more of the active ingredients described herein with other chemical components such as physiologically suitable carriers and excipients. The purpose of a pharmaceutical composition is to facilitate administration of a compound to an organism.

As used herein the term “active ingredient” refers to the dimeric peptides of the present invention accountable for the intended biological effect. It will be appreciated that a polynucleotide encoding a peptide of the present invention may be administered directly into a subject (as is, or part of a pharmaceutical composition) where it is translated in the target cells i.e. by gene therapy. Accordingly, the phrase “active ingredient” also includes such polynucleotides.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference and are further described herein below.

The peptides of the present invention as active ingredients are dissolved, dispersed or admixed in a diluent or excipient that is pharmaceutically acceptable and compatible with the active ingredient as is well known. Suitable carriers or excipients are, for example, water, saline, phosphate buffered saline (PBS), dextrose, glycerol, ethanol, or the like and combinations thereof. Other suitable carriers are well known to those in the art. In addition, if desired, the composition can contain minor amounts of auxiliary substances such as wetting or emulsifying agents, pH buffering agents, stabilizers, binders (e.g., povidone, gelatin, hydroxypropylmethyl cellulose), lubricants, disintegrants (e.g., sodium starch glycollate, cross-linked povidone, cross-linked sodium carboxymethyl cellulose), surface active agents, thickeners, anti-oxidants, and the like.

Pharmaceutical compositions of the present invention may be manufactured by processes well known in the art, e.g., by means of conventional mixing, dissolving, sonicating, granulating, grinding, pulverizing, dragee-making, levigating, emulsifying, encapsulating, entrapping or lyophilizing processes.

Pharmaceutical compositions for use in accordance with the present invention can be formulated in conventional manner using one or more physiologically acceptable carriers or excipients comprising auxiliaries, which facilitate processing of the active ingredients into preparations which, can be used pharmaceutically. Proper formulation is dependent upon the route of administration chosen.

For administration by inhalation, the pharmaceutical compositions according to the present invention can be delivered in the form of an aerosol spray presentation from a pressurized pack or a nebulizer with or without the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator can be formulated containing a powder mix of the peptide and a suitable powder base such as lactose or starch.

For administration into eyes, the pharmaceutical compositions can be delivered as eye drops or eye cream using one or more physiologically acceptable excipients as known in the art.

Pharmaceutical compositions, which can be used orally, include push-fit capsules made of gelatin as well as soft, sealed capsules made of gelatin and a plasticizer such as glycerol or sorbitol. The push-fit capsules can contain the active ingredients in admixture with filler such as lactose, binders such as starches, lubricants such as talc or magnesium stearate and, optionally, stabilizers. In soft capsules, the active compounds can be dissolved or suspended in suitable liquids such as fatty oils, liquid paraffin, or liquid polyethylene glycols. In addition, stabilizers may be added. All formulations for oral administration should be in dosages suitable for the chosen route of administration. For buccal administration, the compositions may take the form of tablets or lozenges formulated in conventional manner.

Dragee cores are provided with suitable coatings. For this purpose, concentrated sugar solutions can be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

For injection, the compounds of the invention can be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Penetrants for example, polyethylene glycol, are generally known in the art.

Pharmaceutical compositions for parenteral administration include aqueous solutions of the active ingredients in water-soluble form. Additionally, suspensions of the active compounds can be prepared as appropriate oily injection suspensions. Suitable natural or synthetic carriers are well known in the art (Pillai et al., Curr. Opin. Chem. Biol. 5, 447, 2001). Optionally, the suspension can also contain suitable stabilizers or agents, which increase the solubility of the active ingredients, to allow for the preparation of highly concentrated solutions. Alternatively, the active ingredient can be in a powder form for reconstitution with a suitable vehicle, e.g., sterile, pyrogen-free water, before use.

The pharmaceutical compositions of the present invention can also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a “therapeutically effective amount” means an amount of a compound effective to prevent, delay, alleviate or ameliorate symptoms of an allergic disease of a subject being treated. Determination of a therapeutically effective amount is well within the capability of those skilled in the art.

Toxicity and therapeutic efficacy of the peptides and analogs, derivatives, or salts thereof described herein can be determined by standard pharmaceutical procedures in cell cultures or in experimental animals, e.g., by determining the IC50 (the concentration which provides 50% inhibition) for a subject peptide. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in The Pharmacological Basis of Therapeutics“, Ch. 1 p. 1).

Depending on the severity of the condition to be treated, dosing can also be a single administration of a slow release composition, with course of treatment lasting from several days to several weeks or until cure is effected or diminution of the disease state is achieved. The amount of a composition to be administered will, of course, be dependent on the immune status and health of the subject being treated, the severity of the disease or condition, the manner of administration, and other relevant factors.

The pharmaceutical composition may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to, a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a peptide composition of the invention formulated in a compatible pharmaceutical carrier may also be prepared, placed in an appropriate container, and labeled for treatment of an indicated condition, as detailed herein.

Thus, according to an embodiment of the present invention, there is provided an article of manufacture which comprises the pharmaceutical composition, as described herein, packaged in a packaging material and identified in print, in or on the packaging material, for use in the treatment of an allergic disorder, as described herein. The formulations and administration methods are intended to be illustrative and not limiting. It will be appreciated that, using the teaching provided herein, other suitable formulations and modes of administration can be readily devised.

It is to be appreciated that peptides are typically less suitable for oral administration due to susceptibility to digestion by gastric acids or intestinal enzymes, however the compositions of the present invention can be administered orally. The pharmaceutical composition of the present invention can also be administered by any suitable means, such as topically, intranasally, subcutaneously, intramuscularly, intravenously, intra-arterially, intraarticularly, intralesionally, parenterally or into the eyes. Administration by inhalation is encompassed in the scope of the present invention.

The dosage of the composition can be administered to the subject in multiple administrations in the course of the treatment period in which a portion of the dosage is administered at each administration.

The peptides of the present invention may be administered as a monotherapy, or in combination with other therapeutic agents, such as, for example, anti-inflammatory agents or steroids. Combination therapies can involve the administration of the pharmaceuticals as a single dosage form or as multiple dosage forms administered at the same time or at different times.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, an and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Peptide Syntheses and characterization

### Peptide Synthesis:

Peptides were synthesized by the solid phase technique utilizing ‘Boc Chemistry’ (Merrifield et al., Biochem. 14: 1385-1390, 1964). Peptides were dissolved in DMSO and stock solutions at a concentration of 20-25 mg/ml were kept at +4° C. In addition similar stock solutions were also prepared in deionized water. The final concentration of the peptides in the assays ranged from 25 μM to 200 μM.

The following monomeric peptides were prepared:

TV5501 (C3a9): DCCNYITR (denoted C3a9) (SEQ ID NO:3)

TV5508 (Monomer A): DCSNYITR (a modification of C3a9 as described herein, in which the second cysteine is modified by serine) (SEQ ID NO:4)

TV5513 (Monomer B): DSCNYITR (a modification of C3a9 as described herein, in which the first cysteine is modified by serine) (SEQ ID NO:5)

To generate dimeric forms of the peptides DSCNYITR and DCSNYITR, peptides were dissolved in DMSO and water at a required concentration, (e.g., 10-20 mg/ml), a 1% iodine solution is used as oxidant and added step-wise, titrating the free thiolate groups. The progress of the reaction was monitored by HPLC. After the reaction was completed, the crude reaction mixtures were purified by preparative HPLC. The structures were verified by MS.

The following dimeric peptides were synthesized:

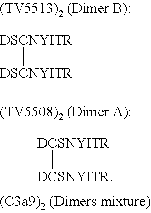

### Characterization:

Water Solubility:

Water solubility was measured by weighing out specific amounts of the peptides and monitoring complete dissolution by the transparency of the solutions. The obtained data is presented in Table 3 below. The values represent relative solubility in deionized water whereby a highest solubility, of 5 mg/ml, has a value of 9 and all other values are with reference thereto.

Circular Dichroism (CD):

In order to assess possible structural features that characterize the active peptides, CD measurement were carried out. The CD measurements were performed on an Applied Photophysics spetrometer in 1 mm light path cell, at room temperature (about 25° C.). The peptides stock solutions were usually of 5 mg/ml in water and the measurements were done at a 100 μg/ml dilution in PBS.

The results are presented in FIGS. 1A-D and further in Table 3 below, and indicate that dimer A, dimer B and monomer B all exhibit the indicated amounts of beta-like structure.

As shown in Table 3, hereinbelow, the highly ordered peptides exhibited enhanced inhibition activity, as compared to partially ordered peptides.

TABLE 3

| | Monomer | Monomer | | | | | |
|---|---|---|---|---|---|---|---|
| | B (5513) | A (5508) | | | Dimers | | Intact |
| | SEQ ID | SEQ ID | Dimer B | Dimer A | Mixture | Monomer | human |
| Assay | NO: 5 | NO: 4 | (5513)₂ | (5508)₂ | (C3a9)₂ | C3a9 | C3a |
| Solubility | 9 | 9 | 9 | 8 | 6-5 | 6-5 | 9 |
| in water | | | | | | | |
| Circular | CD− | CD+ | CD+ | CD+ | ND | ND | CD+ |
| Dichroism | | | | | | | |

### Example 2

### Effect of Dimeric Peptides on IgE-Induced Mast Cell Activation Under In Vitro Conditions

### Materials and Methods

### Secretory Response of Mast Cells:

Mediator secretion by the rat mucosal mast cells, RBL-2H3-line, in response to stimulation by FcεRI clustering was monitored by measuring the activity of the secreted granular enzyme β-hexosaminidase. To this end, a monoclonal DNP-specific murine IgE-class antibody (rat clone 95.3, 1:10³ dilution) was added to 15×10⁶ cells in 10 ml DMEM and incubated in 96 well plates (100 μl suspension/well) for 2 hours. The cell monolayers were then washed three times with Tyrode's buffer (137 mM NaCl, 2.7 mM KCl, 1.8 mM CaCl₂, 0.5 mM MgCl₂, 0.4 mM NaH₂PO₄, 5.6 mM glucose, 10 mM Hepes, 0.1% BSA, pH 7.4.) and stimulated with BSA derivatized by an average of 11 DNP groups per molecule (DNP₁₁-BSA) serving as an antigen.

To study the effect of the peptides on antigen-induced response, RBL-2H3 cells were pre-incubated with various concentrations of the peptides for 5 minutes at room temperature before the antigen was added at the pre-determined suboptimal dose. The peptides were present throughout the assay. After 1 hour incubation at 37 ° C., 25 μl of the cell supernatants were removed and incubated with 50 μl of β-hexosaminidase substrate solution (1.3 mg/ml p-nitrophenyl-N-acetyl-β-D-glucosamine in 0.1 M sodium citrate, pH 4.5) at 37° C. for 45 minutes. The reaction was terminated by the addition of 150 μl of 0.2 M glycine, pH 10.7, and the optical density of the samples was measured at 405 nm. The total enzyme content of the cells/well and the spontaneous enzyme release of the cells/well was measured in each experiment and performed in triplicates.

The total enzyme content of the cells/well (following lysis by triton-x-100) and the spontaneous enzyme release of the cells/well was measured in each experiment. All experiments were performed in triplicate and the results were calculated as net secreted enzyme activity (as fraction or percentage of total cell-content) in response to the stimulus.

Activation of Human Basophils:

The FLOW-CAST Basophil Activation Test, Flow Cytometry Kit (Bühlmann) was used for these experiments. The suboptimal stimulant, an-FcεRI specific mAb (α-chain specific) concentration was first established. To this end, leucocytes of freshly drawn blood were treated with various dilutions of the FcεRI specific mAb. In most of the cases a 40× dilution was used as a suboptimal dose. Cells were incubated with either 100 or 200 μM peptide for 5 minutes at room temperature followed by the addition of the FcεRI-specific mAb. After incubation at 37° C. for 40 minutes, double fluorescent labeling was performed using PE-labeled anti-CD63 and FITC-labeled anti-IgE (all supplied in the FLOW-CAST Basophil Activation Test, Flow Cytometry Kit). After incubation of the samples at 4° C. for 30 minutes, red blood cells were lysed and FACS analysis was performed assessing CD63 expression levels on the IgE⁺ cells.

### Results

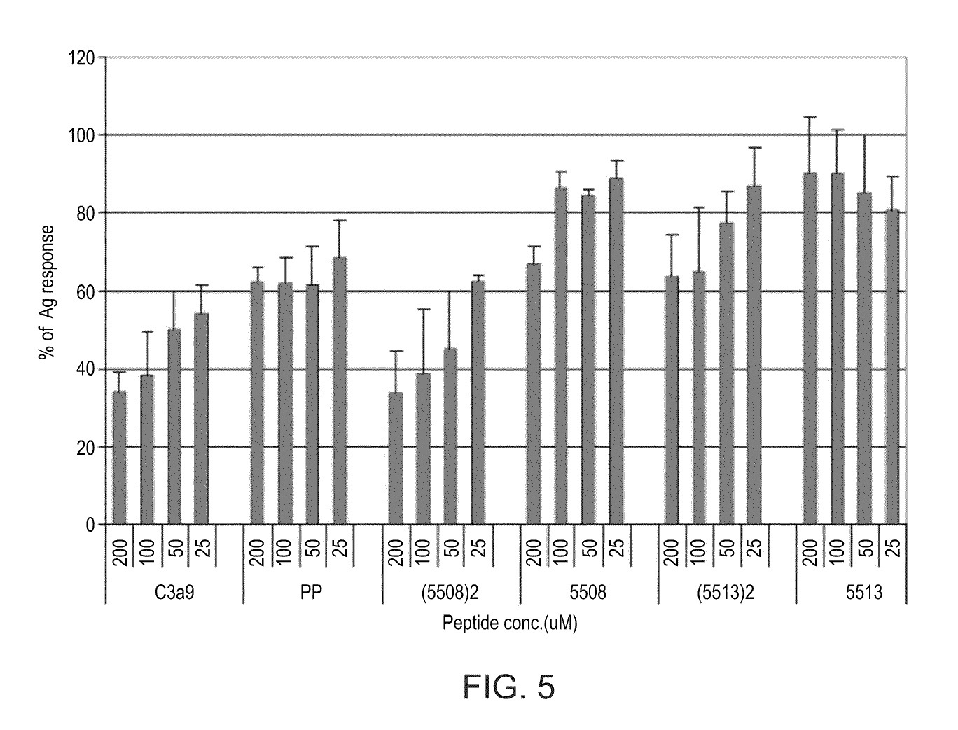

As shown in FIGS. 2A-F and FIG. 5, the dimeric peptide 5508 (Dimer A) exerted similar or higher inhibition as compared with the peptide monomer designated C3a9.

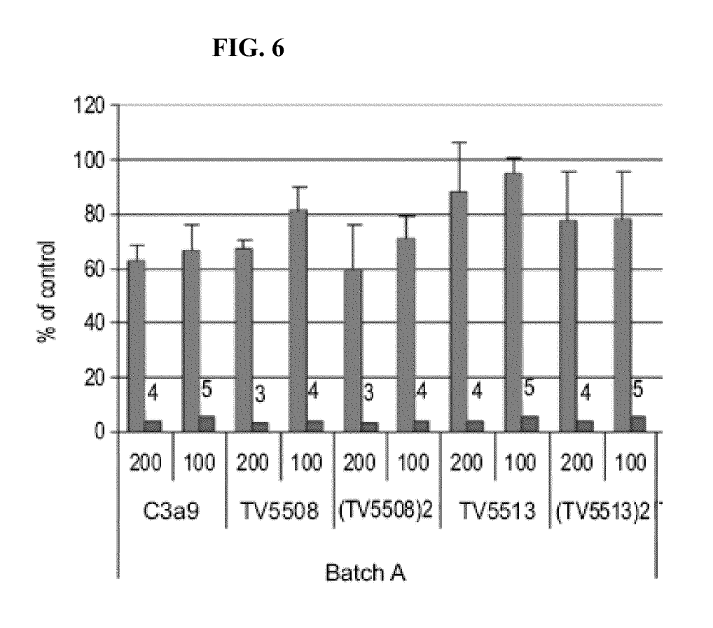

As illustrated in FIG. 3 and FIG. 6, exemplary peptide dimers of the present invention showed enhanced inhibition of CD63 expression as compared to their monomer counterparts.

### Example 3

### Effect of Dimeric Peptides on Passive Systemic Anaphylaxis

### Materials and Methods

Passive Systemic Anaphylaxis:

This assay was performed as reported by J. N. Wu et al., (Journal of Immunology, 2004, 172: 6768-6774). In brief, anesthetized C57 Black mice (4-5/group) were injected with the monoclonal IgE class DNP specific antibody (A2 IgE) by retroorbital injection. A day later, a peptide solution was dripped into the nose of the mice (20 μl of a 500 μm solution). Ten minutes later, animals were challenged with antigen (DNP-BSA₁₁) and 5 minutes later blood was taken for determination of histamine concentration by the Immunotech Histamine-kit.

### Results

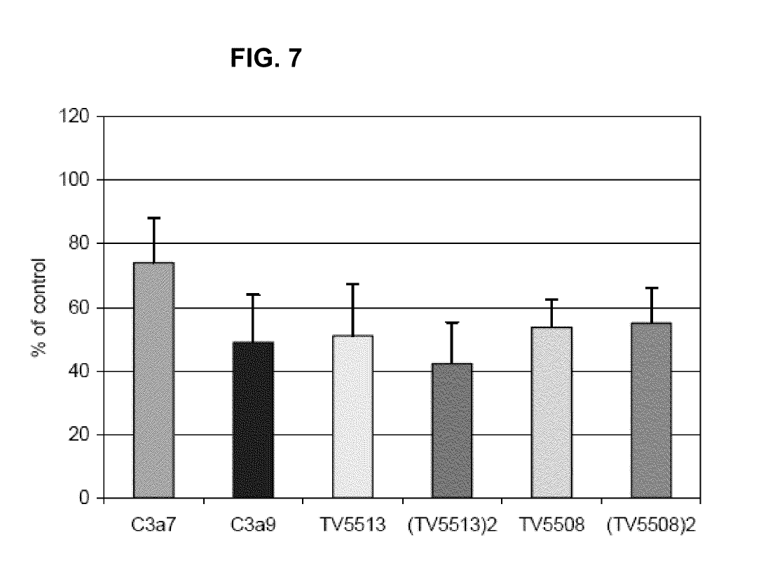

FIGS. 4 and 7 show the extent of inhibition of increase in blood histamine levels in the peptide-pretreated mice following antigen-challenge. As shown in FIGS. 4 and 7 monomeric peptides as well as dimeric peptides showed similar inhibition of histamine release, although dimeric peptide 5513 was found to be more effective.

### Example 4

### Summary of Comparative Data

Table 4 below summarizes the comparative data obtained for the monomeric and dimeric peptides tested.

TABLE 4

| | | Monomer | | | | | |
|---|---|---|---|---|---|---|---|
| | Monomer B | A (5508) | | | Dimers | | Intact |
| | (5513) SEQ | SEQ ID | Dimer B | Dimer A | Mixture | Monomer | human |
| Assay | ID NO: 5 | NO: 4 | (5513)₂ | (5508)₂ | (C3a9)₂ | C3a9 | C3a |
| Solubility in | 9 | 9 | 9 | 8 | 6-5 | 6-5 | 9 |
| water | | | | | | | |
| Circular | CD− | CD+ | CD+ | CD+ | ND | ND | CD+ |
| Dichroism | | | | | | | |
| Inhibition of | No | 75 μg/ml | 25 μg/ml | 15 μg/ml | 150 μg/ml | 200 μg/ml | 2 μg/ml |
| secretion of 3H2 | inhibition | | | | | | |
| of RBL cells | | | | | | | |
| (IC50) | | | | | | | |
| Inhibition of | No | 100 μg/ml | 180 μg/ml | 100 μg/ml | 100 μg/ml | 100 μg/ml | ND |
| degranulation of | inhibition | | | | | | |
| human basophils | | | | | | | |
| Suppression of | 500 μM/ | 500 μM/ | 500 μM/ | 500 μM/ | 500 μM/ | 500 μM/ | ND |
| passive | 20 μl | 20 μl | 20 μl | 0 μl | 20 μl | 20 μl | |
| systemic | | | | | | | |
| anaphylaxix | | | | | | | |
| Mice blood) | | | | | | | |
| (histamine levels | | | | | | | |

The comparative presentation of the data obtained clearly shows (i) a good correlation between circular dichroism and inhibition activity; (ii) improved water solubility and inhibition activity of the dimers formed from monomeric peptides which include a single cysteine residue (monomeric peptides derived from C3a9 in which one Cys was replaced by Ser) as compared to the dimers formed from monomeric peptides which include 2 Cys residues (as in C3a9), and which were not formed by directing the position of formed disulfide bridge; and (iii) improved inhibition activity of the dimers formed from monomeric peptides as compared to the monomeric peptides.

Overall, these results indicate superior properties and activity of dimeric peptides according to embodiments of the invention.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-40. (canceled)

41. A peptide composition comprising a dimeric peptide, said dimeric peptide comprising two peptide monomers, each independently comprising the amino acid sequence:
Asp-X1-X2-Asn-Tyr-Ile-Thr-X3
wherein:
X1 is selected from the group consisting of Cys and a Cys derivative;
X2 is selected from the group consisting of Cys and a Cys derivative;
and
X3 is selected from the group consisting of Arg and Glu-Leu-Arg, provided that at least one of X1 and X2 is Cys,
wherein a mol percentage of said dimeric peptide in the composition is at least 50 mol percents.

42. The peptide composition of claim 41, wherein at least one of said peptide monomers comprises said amino acid sequence in which one of X1 and X2 is said Cys derivative.

43. The peptide composition of claim 41, wherein said Cys derivative is devoid of a free thiol group.

44. The peptide composition of claim 43, wherein said Cys derivative is selected from the group consisting of a protected Cys and Ser.

45. The peptide composition of claim 43, wherein said Cys derivative is Ser.

46. The peptide composition of claim 45, wherein each said peptide monomers independently has an amino acid sequence selected from the group consisting of Asp-Cys-Ser-Asn-Tyr-Ile-Thr-Arg as set forth in SEQ ID NO:4; and
Asp-Ser-Cys-Asn-Tyr-Ile-Thr-Arg as set forth in SEQ ID NO:5.

47. The peptide composition of claim 41, wherein at least one of said peptide monomers comprises said amino acid sequence in which each of X1 and X2 is Cys.

48. The peptide composition of claim 47, wherein each of said peptide monomers comprises the amino acid sequence Asp-Cys-Cys-Asn-Tyr-Ile-Thr-Arg as set forth in SEQ ID NO:3.

49. The peptide composition of claim 41, wherein said two peptide monomers are linked to one another by at least one disulfide bond.

50. The peptide composition of claim 41, wherein said two peptide monomers are linked to one another by a single disulfide bond.

51. The peptide composition of claim 49, wherein said disulfide bond is an intermolecular disulfide bond formed between two of said Cys residues.

52. The peptide composition of claim 41, wherein at least one of said two peptide monomers consists of said amino acid sequence.

53. The peptide composition of claim 41, wherein a mol percentage of said dimeric peptides in the composition is at least 90 mol percents.

54. The peptide composition of claim 41, wherein a mol percentage of said dimeric peptides in the composition is at least 99 mol percents.

55. The peptide composition of claim 41, wherein a mol percentage of a multimeric peptide, a monomeric peptide and/or a dimeric peptide other than said dimeric peptide is lower than 1 mol percent.

56. A process of preparing the peptide composition of claim 41, the process comprising:
reacting two monomeric peptides, each independently comprising the amino acid sequence:
Asp-X1-X2-Asn-Tyr-Ile-Thr-X3
wherein:
X1 is selected from the group consisting of Cys and a Cys derivative;
X2 is selected from the group consisting of Cys and a Cys derivative;
and
X3 is selected from the group consisting of Arg and Glu-Leu-Arg, provided that at least one of X1 and X2 is Cys,
in the presence of an oxidizing agent, thereby producing the peptide composition.

57. The process of claim 56, wherein said reacting is performed under conditions that favor intermolecular interactions between said monomeric peptides.

58. The process of claim 56, wherein at least one of said monomeric peptides comprises an amino acid sequence in which one of X1 and X2 is a Cys derivative.

59. The process of claim 58, wherein said Cys derivative is Ser.

60. The process of claim 58, wherein said Cys derivative is a protected Cys.

61. The process of claim 60, further comprising, prior to reacting said two monomeric peptides with said oxidizing agent:
reacting a monomeric peptide which comprises an amino acid sequence in which X1 and X1 are each Cys with a cysteine protecting group, to thereby obtain said monomeric peptide is which each of X1 and X2 is said protected Cys.

62. The process of claim 61, further comprising, prior to reacting said two monomeric peptides with said oxidizing agent:
selectively removing said cysteine protecting group, to thereby obtain a monomeric peptide is which one of X1 and X2 is said protected Cys.

63. The process of claim 60, further comprising, prior to reacting said two monomeric peptides with said oxidizing agent:
reacting a monomeric peptides which comprises an amino acid sequence in which X1 and X1 are each Cys with a cysteine protecting group, under conditions that favor selective protection, to thereby obtain said peptide monomer is which one of X1 and X2 is said protected Cys.

64. The process of claim 56, wherein said oxidizing agent is iodine.

65. A pharmaceutical composition comprising the peptide composition of claim 41, and a pharmaceutically acceptable carrier.

66. The pharmaceutical composition of claim 65, being packaged in a packaging material an identified in print, in or on, said packaging material, for use in the treatment of an allergic disorder.

67. The pharmaceutical composition of claim 66, wherein the allergic disorder is selected from the group consisting of allergic rhinitis, pulmonary diseases, allergic dermatosis, allergic conjunctivitis, gastrointestinal allergies, cramping, nausea, vomiting, diarrhea, irritable bowel disease, ophthalmic allergies, cheilitis, vulvitis, and anaphylaxis.

68. The pharmaceutical composition of claim 65, wherein at least one of said peptide monomers comprises said amino acid sequence in which one of X1 and X2 is said Cys derivative.

69. The pharmaceutical composition of claim 65, wherein said Cys derivative is devoid of a free thiol group.

70. The pharmaceutical composition of claim 69, wherein said Cys derivative is selected from the group consisting of a protected Cys and Ser.

71. The pharmaceutical composition of claim 70, wherein each said peptide monomers independently has an amino acid sequence selected from the group consisting of Asp-Cys-Ser-Asn-Tyr-Ile-Thr-Arg as set forth in SEQ ID NO:4; and Asp-Ser-Cys-Asn-Tyr-Ile-Thr-Arg as set forth in SEQ ID NO:5.

72. The pharmaceutical composition of claim 65, wherein at least one of said peptide monomers comprises said amino acid sequence in which each of X1 and X2 is Cys.

73. The pharmaceutical composition of claim 72, wherein each of said peptide monomers comprises the amino acid sequence Asp-Cys-Cys-Asn-Tyr-Ile-Thr-Arg as set forth in SEQ ID NO:3.

74. The pharmaceutical composition of claim 65, wherein said two peptide monomers are linked to one another by at least one disulfide bond.

75. The pharmaceutical composition of claim 74, wherein said disulfide bond is an intermolecular disulfide bond formed between two of said Cys residues.

76. The pharmaceutical composition of claim 65, wherein at least one of said two peptide monomers consists of said amino acid sequence.

77. The pharmaceutical composition of claim 65, wherein a mol percentage of a multimeric peptide, a monomeric peptide and/or a dimeric peptide other than said dimeric peptide is lower than 1 mol percent.

78. A method of treating an allergic disorder comprising administering to a subject in need thereof a therapeutically effective amount of the peptide composition of claim 41, thereby treating the allergic disorder.

79. The method of claim 78, wherein the allergic disorder is selected from the group consisting of allergic rhinitis, pulmonary diseases, allergic dermatosis, allergic conjunctivitis, gastrointestinal allergies, cramping, nausea, vomiting, diarrhea, irritable bowel disease, ophthalmic allergies, cheilitis, vulvitis, and anaphylaxis.


SEQUENCE LISTING (7 sequences)

SEQ ID NO: 1 - protein, 77 aa, Homo sapiens
  misc_feature: Human complement component C3a
SVQLTEKRMDKVGKYPKELRKCCEDGMRENPMRFSCQRRTRFISLGEACKKVFLDCCNYITELRRQHARASHLGLAR

SEQ ID NO: 2 - protein, 10 aa, Artificial sequence
  C3a Derived synthetic peptide (C3a7)
DCCNYITELR

SEQ ID NO: 3 - protein, 8 aa, Artificial sequence
  C3a Derived synthetic peptide (C3a9)
DCCNYITR

SEQ ID NO: 4 - protein, 8 aa, Artificial sequence
  Modified C3a derived synthetic peptide
DCSNYITR

SEQ ID NO: 5 - protein, 8 aa, Artificial sequence
  Modified C3a derived synthetic peptide
DSCNYITR

SEQ ID NO: 6 - protein, 8 aa, Artificial sequence
  Modified C3a derived synthetic peptide
DCTNYITR

SEQ ID NO: 7 - protein, 8 aa, Artificial sequence
  Modified C3a derived synthetic peptide
DTCNYITR